(19) United States
(12) Patent Application Publication
(10) Pub. No.: US 2005/0009132 A1
(43) Pub. Date: Jan. 13, 2005

(54) Culture medium for detecting and/or discriminating enterococcus and method therefor

(76) Inventor: Alain Rambach, Paris (FR)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/481,004

(22) PCT Filed: Jun. 13, 2002
(86) PCT No.: PCT/FR02/02025

(30) Foreign Application Priority Data
Jun. 13, 2001 (FR) 01/07730

(51) Int. Cl. C12Q 1/04; C12Q 1/16

(52) U.S. Cl. 435/34; 435/35

(57) ABSTRACT

The invention concerns a culture medium for isolating enterococcus comprising violet crystal, and preferably gram-negative bacteria inhibitors and chromogens. The invention also concerns a method for detecting enterococcus using said medium.

The present invention relates to a chromogenic culture medium intended for identifying enterococci.

In the clinical field, the detection of enterococci is very important because of the appearance of strains which are resistant to antibiotics, in particular to vancomycin, the antibiotic most widely used for treating infections. These nosocomial infections can endanger the lives of the patients affected.

Streptococci isolated from the intestine or group D streptococci were conventionally distinguished as two groups according to their physiological characteristics:
“enterococci” (Streptococcus faecium and Streptococcus faecalis) capable of growing under hostile conditions;
“non enterococci group D streptococci” (Streptococcus bovis and Streptococcus equinus) incapable of multiplying under hostile conditions.

In 1982 and 1983, genomic studies (DNA—rRNA hybridizations, the establishment of dictionaries of oligonucleotides of 16S rRNA) showed that “enterococci” and “non enterococcal group D streptococci” belonged to two different genera.

In 1984, Schleifer and Kilpper-Bälz, while studying the results of DNA-23S rRNA hybridizations and DNA-DNA hybridizations confirmed the earlier results and these two authors proposed transferring the “enterococcal group of streptococci” to the genus enterococcus which is distinct from the genus streptococcus (Int. J. Syst. Bacteriol., 1984, 34, 31-34).

A very good study on enterococci can be found at the site www.bacterio.cict.fr/bacdico/ee/enterococcus.html, or at the site www.life.umd.edu/classroom/bsci424/PathogenDescriptions/Enterococcus.htm, or at the site www.enterococcus.ouhsc.edu/lab methods.asp.

It is therefore important to have a reliable and quick test which makes it possible to detect contaminations by these bacteria, which test should be both sensitive and specific.

Enterococci generally grow in temperature ranges from 10° to 45° C., the optimum growth being around 35° C., on non-selective medium (blood agar or chocolate agar). However, such a medium also allows the growth of other bacteria and does not make it possible to effectively distinguish enterococci.

There are now certain culture media for the detection of enterococci, in particular the MacConkey Agar medium, without Crystal Violet, sold by Difco. The Crystal Violet is a compound which inhibits the growth of Gram-positive bacteria, which is considered to inhibit the growth of enterococci and of staphylococci (it should be recalled that enterococci are Gram-positive cocci which appear in isolation or in pairs or in short chains). The “Difco Manual”, 11th edition, page 288, indicates “MacConkey Agar w/o CV (Crystal Violet) is a differential medium that is less selective than MacConkey Agar. The lack of crystal violet permits the growth of Staphylococcus and Enterococcus”.

This medium also contains other growth inhibitors such as bile salts. These other inhibitors are effective for inhibiting the growth of Gram-positive bacteria, but not enterococci.

Indeed, enterococcal bacteria can also grow in media containing bile salts, the colonies not dissolving after exposure to bile. It is also possible to grow enterococcal bacteria in a medium containing an NaCl concentration of 6.5%, the streptococci not possessing this property.

From these observations that media for enterococci do not contain Crystal Violet, but contain other inhibitors instead, it can be concluded that, under numerous conditions, Crystal Violet is an inhibitor of enterococcal growth. This hypothesis is reinforced by the fact that among the various MacConkey media, the one used to detect enterococci does not contain Crystal Violet, but contains bile salts. However, this medium also allows the growth of staphylococci.

It should be noted that the method most commonly used for the detection of enterococci consists of a filtration technique on a selective medium (such as the m-Enterococcus agar medium=Slanetz and Bartley medium or oxolinic acid-esculin-azide medium=OAA medium) followed by confirmation by culture on bile-esculin agar incubated for 48 hours at 44° C. The selective effect may be reinforced by incubating the Slanetz and the Bartley medium at 41° C. Most often, complete identification is not obtained and only the identification of catalase is carried out in order to eliminate certain staphylococcal strains which are capable of developing on the media used.

It is therefore advisable to have a medium allowing the detection of enterococci and which also makes it possible to differentiate them from staphylococci, which grow in general on culture media for enterococci.

In the context of the present invention, it has been shown that it is possible to adjust the concentration of Crystal Violet such that it retains its role of growth inhibition for most Gram-positive bacteria, and in particular staphylococci, while allowing the growth of enterococci.

Thus, the prior art media (in particular the MacConkey Agar medium sold by Difco Laboratories) generally use a Crystal Violet concentration equal to 1 mg/l. In the context of the present invention, it has been shown that the addition of Crystal Violet at a concentration of 0.1 up to about 1.5 mg/l makes it possible to maintain the growth of enterococci, in particular when the medium does not contain other growth inhibitors for Gram-positive bacteria.

It has also been shown that the addition of Crystal Violet makes it possible to avoid using sodium azide which is very often present in enterococci detection media (for its properties of inhibiting the growth of certain bacteria, but which is toxic for humans). Preferably, a medium according to the invention will not therefore contain sodium azide.

This result, and in particular the fact that the Crystal Violet concentration may be greater than 1 mg/l under certain conditions while allowing the growth of enterococci, could not have been predicted in the light of the knowledge described in the prior art, in particular the fact that it is also possible to eliminate sodium azide.

Thus, the subject of the present invention is a culture medium for detecting and/or distinguishing enterococci, characterized in that it contains, in a culture medium for enterococci, Crystal Violet at a concentration allowing the growth of enterococci and the inhibition of the growth of most Gram-positive bacteria.

Preferably, said concentration is greater than 0.1 mg/l, most preferably greater than 0.25 mg/l, or greater than 0.5 mg/l. Thus, inhibition of the growth of a large quantity of Gram-positive bacteria is observed after addition of Crystal Violet at a concentration as low as 0.1 mg/l.

In a particular embodiment of the invention, said concentration is less than 1.5 mg/l, more preferably less than 1 mg/l. When the Crystal Violet concentration is about 1 mg/l or greater than this value, it is then advisable to reduce, or even eliminate the other inhibitors of Gram-positive bacteria, in particular the bile salts. It is within the capability of persons skilled in the art to adjust the concentrations of inhibitors according to the concentration of Crystal Violet added to the medium according to the invention. In another embodiment of the invention, the Crystal Violet concentration is less than 0.8 mg/l, more preferably less than 0.7 mg/l. At such concentrations, it is possible to optionally add other inhibitors of Gram-positive bacteria.

A person skilled in the art knows what the term “culture medium for enterococci” means, that is to say a culture medium containing the nutrients necessary to allow the growth of these bacteria. There may be mentioned in particular peptone from casein, from soybean, from meat, from yeast extract or from beef, dextrose and the like.

Preferably, the medium according to the invention further comprises at least one chromogenic agent, a substrate for an enzyme for fermenting sugars, said enzyme being preferably a glucosidase, in particular β-glucosidase or a galactosidase, in particular β-galactosidase.

The fact that it is possible to add a chromogenic agent is completely unexpected since Crystal Violet already colors the media according to the invention, which therefore dissuades from adding a constituent providing color, such as a chromogenic agent.

Preferably, said chromogenic agent releases, by hydrolysis, a precipitable chromophore chosen from indoxyl, haloindoxyl (bromoindoxyl, chloroindoxyl, fluoroindoxyl, iodoindoxyl, dichloroindoxyl, chlorobromoindoxyl, trichloroindoxyl), methylindoxyl or hydroxyquinoline derivatives, in particular the following derivatives: 6-chloroindoxyl, 5-bromoindoxyl, 3-bromoindoxyl, 6-fluoroindoxyl, 5-iodoindoxyl, 4,6-dichloroindoxyl, 6-7-dichloroindoxyl, 5-bromo-4-chloroindoxyl, 5-bromo-6-chloroindoxyl, 4,6,7-trichloroindoxyl, N-methylindoxyl or 8-hydroxyquinoline.

Preferably, said chromogenic substrate for β-glucosidase is an indoxylglucoside, in particular 5-bromo-4-chloro-3-indoxyl-β-glucoside and/or said chromogenic agent, a substrate for β-galactosidase, is an indoxylgalactoside, in particular 5-bromo-6-chloro-3-indoxyl-β-galactoside.

In order to better allow the detection of enterococci, it is also possible to add, to the culture medium according to the invention, growth inhibitors for Gram-negative bacteria, such as nalidixic acid or colistin.

To detect atypical enterococci which are resistant to certain antibiotics, and which are largely responsible for nosocomial infections, it is also possible to add said antibiotics to the media according to the invention. Vancomycin will thus be added at a concentration of about 6 mg/l. It should be noted that it is possible to detect the proportion of resistant bacteria in a sample by plating on a dish without antibiotics and a dish containing them and by comparing the number of colonies identified as enterococci on each dish.

The invention also relates to the use of a culture medium according to the invention for detecting and/or distinguishing enterococci, and to a method for detecting and/or distinguishing enterococci in a sample, characterized in that it comprises the steps consisting in:
a. inoculating a culture medium according to the invention with said sample of an inoculum derived from the sample,
b. detecting the presence of enterococci on said culture medium.

The presence of enterococci is detected by the growth of the colonies on the medium, and this is helped by their coloration after releasing the chromophore from the substrate chromogen for the enzyme.

A chromophore would be preferably chosen which has a wavelength different from the wavelength of Crystal Violet so as to identify it more easily. However, the medium according to the invention also allows the use and the detection of chromophores having a wavelength close to the wavelength of Crystal Violet.

In general, the invention also relates to a culture medium for the detection of Gram-positive or Gram-negative bacteria comprising, in addition to growth factors for said Gram-positive or Gram-negative bacteria, a chromogenic agent and Crystal Violet, the Crystal Violet being present at a concentration allowing the growth of said bacteria which it is sought to detect and the differential inhibition of the growth of Gram-positive bacteria, said concentration being preferably between 0.1 and 1.5 mg/l.

The invention also relates to the use of Crystal Violet as a growth-selective inhibitor for the preparation of a culture medium for the detection of Gram-positive or Gram-negative bacteria, additionally containing a chromogenic agent.

The invention has therefore demonstrated that it is possible to add a colorant to a chromogenic medium, while retaining the chromogenic properties.

The chromogen is chosen such that the chromophore released has a wavelength different from that of Crystal Violet or the colorant used in the chromogenic medium.

### EXAMPLES

### Example 1

A preferred medium for carrying out the invention comprises (for one liter):

| | |
|---|---|
| Agar | 15 g |
| Yeast extract and peptones | 9 g |
| NaCl | 5 g |
| Nalidixic acid | 50 mg |
| Colistin | 5 mg |
| Crystal Violet | 0.5 mg |
| 5-bromo-4-chloro-3-indoxyl-β-glucoside | 50 mg |

### Example 2

Plating of bacteria on the medium according to the invention gave the following results (24 hours of incubation at 37° C.).

| | Growth | Color |
|---|---|---|
| Enterococci | + | Mauve-blue |
| Staphylococci | − | — |

The use of the medium according to the invention therefore makes it possible to detect the enterococci, and to distinguish them from staphylococci.

### Example 3

Examples of media for enterococci which may be used in the context of the present invention, by adding Crystal Violet thereto and by optionally removing sodium azide therefrom, or by adding chromogenic agents, and optionally vancomycin, for detecting resistant strains.

Bile-esculin medium (composition in grams by liter):

| | |
|---|---|
| Meat extract: | 3.0 |
| Meat peptone: | 5.0 |
| Beef bile: | 40.0 |
| Esculin: | 1.0 |
| Iron citrate: | 0.5 |
| Agar: | 14.5 |

This medium may be enriched with 5% horse serum.

Bile-esculin-azide medium (composition in grams by liter):

| | |
|---|---|
| Tryptone: | 17.0 |
| Peptone: | 3.0 |
| Yeast extract: | 5.0 |
| Esculin: | 1.0 |
| NaCl: | 5.0 |
| Ammoniacal iron citrate: | 0.5 |
| Sodium citrate: | 1.0 |
| Sodium azide: | 0.25 |
| Beef bile: | 10.0 |
| Agar: | 13.5 |

Slanetz and Bartley medium or m-Enterococcus agar (composition in grams by liter):

| | |
|---|---|
| Tryptone: | 15.0 |
| Peptone: | 5.0 |
| Yeast extract: | 0.5 |
| Glucose: | 2.0 or 5.0 |
| K₂HPO₄: | 4.0 |
| Sodium azide: | 0.4 |
| 2,3,5-triphenyltetrazolium chloride | 10.0 |
| (solution at 1 percent): | |
| Agar: | 10 |

Oxolinic acid-esculin-azide medium or OAA medium (composition in grams per liter):

| | |
|---|---|
| Tryptone: | 20.0 |
| Yeast extract: | 5.0 |
| Glucose: | 1.0 |
| NaCl: | 5.0 |
| Ammoniacal iron citrate: | 0.5 |
| Sodium citrate: | 1.0 |
| Sodium azide: | 0.4 |
| Oxolinic acid: | 0.005 |
| Agar: | 10.0 |

Esculin-azide-kanamycin agar (composition in grams per liter)

| | |
|---|---|
| Tryptone: | 20.0 |
| Yeast extract: | 5.0 |
| Glucose: | 1.0 |
| NaCl: | 5.0 |
| Ammoniacal iron citrate: | 0.5 |
| Sodium citrate: | 1.0 |
| Sodium azide: | 0.15 |
| Kanamycin sulfate: | 0.02 |
| Agar: | 10.0 |

1. A culture medium for detecting and/or distinguishing enterococci, characterized in that it contains, in a culture medium for enterococci, Crystal Violet at a concentration allowing the growth of enterococci and the inhibition of the growth of most Gram-positive bacteria, said concentration being between 0.1 and 1.5 mg/l.

2. The culture medium as claimed in claim 1, characterized in that it additionally comprises at least one chromogenic agent, a substrate for an enzyme for sugar fermentation.

3. The medium as claimed in claim 2, characterized in that said enzyme is a glucosidase, in particular β-glucosidase or a galactosidase, in particular β-galactosidase.

4. The culture medium as claimed in claim 2 or 3, characterized in that said chromogenic agent releases, by hydrolysis, a precipitable chromophore chosen from indoxyl, haloindoxyl (bromoindoxyl, chloroindoxyl, fluoroindoxyl, iodoindoxyl, dichloroindoxyl, chlorobromoindoxyl, trichloroindoxyl), methylindoxyl or hydroxyquinoline derivatives, in particular the following derivatives: 6-chloroindoxyl, 5-bromoindoxyl, 3-bromoindoxyl, 6-fluoroindoxyl, 5-iodoindoxyl, 4,6-dichloroindoxyl, 6-7-dichloroindoxyl, 5-bromo-4-chloroindoxyl, 5-bromo-6-chloroindoxyl, 4,6,7-trichloroindoxyl, N-methylindoxyl or 8-hydroxyquinoline.

5. The culture medium as claimed in claim 3 or 4, characterized in that said β-glucosidase substrate is an indoxylglucoside, and/or said β-galactosidase substrate is an indoxyl-galactoside.

6. The culture medium as claimed in claim 5, characterized in that the β-glucosidase substrate is 5-bromo-4-chloro-3-indoxyl-β-glucoside and/or the β-galactosidase substrate is 5-bromo-6-chloro-3-indoxyl-β-galactoside.

7. The culture medium as claimed in one of claims 1 to 6, characterized in that it also contains growth inhibitors for Gram-negative bacteria.

8. The culture medium as claimed in one of claims 1 to 7, characterized in that it comprises (for one liter):

| | |
|---|---|
| Agar | 15 g |
| Yeast extract and peptones | 9 g |
| NaCl | 5 g |
| Nalidixic acid | 50 mg |
| Colistin | 5 mg |
| Crystal Violet | 0.5 mg |
| 5-bromo-4-chloro-3-indoxyl-β-glucoside | 50 mg |

9. The culture medium as claimed in one of claims 1 to 8, additionally containing antibiotics.

10. The culture medium as claimed in one of claims 1 to 9, characterized in that it does not contain sodium azide.

11. The use of a culture medium as defined in one of claims 1 to 10 for detecting and/or distinguishing enterococci.

12. A method for detecting and/or distinguishing enterococci in a sample, characterized in that it comprises the steps consisting in:
a. inoculating a culture medium as defined in one of claims 1 to 10 with said sample of an inoculum derived from the sample,
b. detecting the presence of enterococci on said culture medium.

13. A culture medium for the detection of Gram-positive or Gram-negative bacteria comprising, in addition to growth factors for said Gram-positive or Gram-negative bacteria, a chromogenic agent and Crystal Violet, the Crystal Violet being present at a concentration allowing the growth of said bacteria which it is sought to detect and the differential inhibition of the growth of Gram-positive bacteria, said concentration being between 0.1 and 1.5 mg/l.

14. The use of Crystal Violet as a growth-selective inhibitor for the preparation of a culture medium for the detection of Gram-positive or Gram-negative bacteria, additionally containing a chromogenic agent.
